(19) United States
(12) Patent Application Publication
(10) Pub. No.: US 2012/0323084 A1
(43) Pub. Date: Dec. 20, 2012

(54) SET OF BLOCKS AND METHODS OF PLAY AND DIAGNOSIS

(76) Inventors: Geoffrey S. FROST, Toronto (CA); Thuvatahan SIVAYOGAN, Toronto (CA); Malgosia PAKULSKA, Toronto (CA); Matthew J. CAICCO, Toronto (CA); Nimalan THAVANDIRAN, Toronto (CA); Ajmal KHAN, Toronto (CA)

(21) Appl. No.: 13/324,603

(22) Filed: Dec. 13, 2011

Related U.S. Application Data
(60) Provisional application No. 61/422,583, filed on Dec. 13, 2010.

(51) Int. Cl. A61B 5/00; A63H 33/04

(52) U.S. Cl. 600/300; 446/85

(57) ABSTRACT

A set of blocks, for playing a game comprises a plurality of blocks, each block having one characteristic selected from the first category and one characteristic selected from the second category. At least some of the characteristics in the first category are different from one another and at least some of the characteristics in the second category are different from one another. Categories can include a degree of abstraction and may include shape, size, colour, texture, weight and surface finish. The blocks can be provided with a game board and a die, for playing a game or for diagnosis. The blocks can be used for play, and for diagnosis of children with autism or other individuals with other neurological disorders.

### FIELD

This invention relates to a game, a method of playing a game that may be applicable to children with neurological disorders, a method of diagnosis, and to a set of blocks.

### BACKGROUND

The following paragraphs are not an admission that anything discussed in them is prior art or part of the knowledge of persons skilled in the art.

There are a wide variety of neurological disorders that affect children, and additionally there are children and individuals that have cognitive learning challenges/neurological disorders, which may be difficult to categorize (e.g. dyslexia, visual memory). One neurological disorder is autism, which is a complex neurological disorder characterized by impaired social interaction, and difficulties in communication. Difficulties in communication may stem from the inability to generalize information across different experiences. As a result of the inability to generalize information, autistic individuals have difficulty in categorizing ideas.

Categorization is the process that is most often used to generalize information across situations in normal individuals. There is substantial evidence that autistic children have difficulty with categorization. The inability of autistic children to categorize is exploited in the Wisconsin Card Sorting Test, a widely used autism diagnostic test. This test asks the participant to sort a set of cards according to a category, which can be one of the shape, number and color of individual shapes on each card. The category by which sorting is to occur is chosen by the examiner, who does not inform the participant of the selected category. A participant is continually given feedback as to whether they are sorting the cards correctly. As time progresses, the examiner will change the sorting category.

Development of categorization skills is believed to be essential in developing normal social function. Essential to this is the development of the ability to understand abstract concepts. It is difficult to discuss baseball, for instance, if an individual's definition and knowledge of sport is defined as ice hockey. Generalizing the concept of sport to beyond ice hockey would allow individuals to discuss, understand, and participate in a wider range of physical and social activities.

Beyond the theoretical, autistic children often have a natural tendency to sort or stack objects, and blocks have been found to be an ideal toy for autistic children. Regular blocks, however, can only be categorized in a limited number of ways. Further, while there are a large number of different sets of blocks available, as noted above, typically the characteristics or categories likely to be assigned to the blocks are limited, e.g. to shape and size. It may be desirable to provide block sets that have a greater variety of categories, and it is believed that this would be attractive or useful, not only for use with children with autism, but for individuals with normal development. It may also be desirable to provide a tool, such as a set of blocks that can be used for diagnosis and rehabilitation, with individuals with other neurological disorders.

### SUMMARY

The following introduction is intended to introduce the reader to this specification but not to define any invention. One or more inventions may reside in a combination or sub-combination of the apparatus elements or method steps described below or in other parts of this document. The inventor does not waive or disclaim his rights to any invention or inventions disclosed in this specification merely by not describing such other invention or inventions in the claims.

This invention relates to a set of game of pieces and a method of playing a game, more particularly, but not exclusively the invention relates to the fields of (a) neurological disorder diagnosis, (b) neurological disorder management, (c) neurological disorder treatment/rehabilitation , and (d) a set of game pieces and method for use by people with a neurological disorder.

The invention provides a set of blocks, which may be made from wood, designed for use by children with neurological disorders, their parents, and their caregivers. The present invention also provides a single or multi-player game, played using the set of blocks. The set of blocks may include a variety of different characteristics for categorizing the blocks, e.g.: (a) shape, (b) size, (c) colour, (d) finish, (e) texture, (f) weight. The categories of the blocks have been presented from lest abstract to most abstract, and the most abstract category, weight, cannot be determined by sight alone.

The blocks and the associated game can develop the ability of abstract thought within individuals with neurological disorders.

In accordance with a first aspect of the present invention, there is provided a set of blocks, for playing a game or for diagnosis, the set of blocks comprising a plurality of blocks, each block having one characteristic selected from a first category and one characteristic selected from a second category, wherein at least some of the characteristics in the first category are different from one another, and at least some of the characteristics in the second category are different from one another.

The set of blocks can include at least one characteristic from at least three or six different categories. For each of the categories present, there are at least two different instances of that category.

The present invention also provides a method of playing a game, preferably for a child with a neurological disorder, the method comprising:

providing a plurality of blocks as defined above and a die bearing on its faces an indication of each of the different categories of features of the blocks;

rolling the die, to select a category, or otherwise randomly selecting one of the categories; and

asking the player of the game to sort the blocks according to the selected category.

The present invention further provides a method of diagnosing autism in a child, the method comprising:

providing a plurality of blocks as defined above;

asking the child to sort the blocks according to an unspecified category, comprising a sort criterion;

providing feedback to the child as to whether blocks are sorted correctly or not; and

periodically changing the category by which blocks are to be sorted, while continuing to provide feedback as to whether the blocks are sorted correctly or not;

and noting mistakes made in sorting and how long it takes the child to learn a new sort criterion.

A further method of the present invention provides for the playing of a game with two people, preferably two children with neurological disorders, the method comprising providing a plurality of blocks as defined above, dividing the blocks between the two players, and providing a die bearing on its faces an indication of each of the different categories of features of the blocks;

having a first player select and place a block on a playing surface;

rolling the die, to select a category, or otherwise randomly selecting one of the categories, and the second player then playing a block by placing it on the playing surface adjacent the first block, with the second block having the same feature from the selected category as the first block;

repeating the last step for each player in turn

### BRIEF DESCRIPTION OF THE DRAWING FIGURES

For a better understanding of the present invention and to show more clearly how it may be carried into effect, reference will now be made, by way of example, to the accompanying drawings which show, by way of example, the present invention and in which:

FIGS. 1A, 1B and 1C show perspective, side and plan views a cylindrical block, and FIG. 1D shows a perspective view of a different size of cylindrical block;

FIGS. 2A, 2B, 2C and 2D show perspective, front, side and plan views of an hexagonal block, and FIG. 2D shows a perspective view of a different size of hexagonal block;

FIGS. 3A, 3B and 3C show perspective, side and plan views a block with the shape of a rectangular prism, and FIG. 3D shows a perspective view of a different sized block with the shape of a rectangular prism;

FIGS. 4A, 4B and 4C show perspective, side and plan views a block with a square cross section, and FIG. 4D shows a perspective view of a different size of block with a square cross section;

FIGS. 5A, 5B and 5C show perspective, side and plan views of a block with a pyramid shape, and FIG. 5D shows a perspective view of a different size of block with a pyramid shape;

FIGS. 6A, 6B and 6C show perspective, side and plan views of a spherical block, and FIG. 6D shows a perspective view of a different size of spherical block;

FIGS. 7A, 7B and 7C show perspective, side and plan views of a block with a shape of a triangular prism, and FIG. 7D shows a perspective view of a different size of block with a shape of a triangular prism;

FIGS. 8A, 8B, 8C and 8D show perspective, front, side and plan views of a block with an elliptical cross section, provided with a bore for a weight;

FIGS. 9A, 9B, 9C and 9D show perspective, front, side and plan views of a solid weight with an elliptical cross section, for use in the block of FIG. 8;

FIGS. 10A, 10B, 10C and 10D show perspective, front, side and plan views of a plug with an elliptical cross section, for use in the block of FIG. 8;

FIG. 11 shows schematically assembly of the block of FIG. 8 with a weight and a plug; and

FIGS. 12A, 12b, 12C, 12D, and 12E show perspective, front, side, an opposite side and plan views of an element for forming a block with a profile of the block of FIG. 8.

FIGS. 13A-G show perspective views of a cubic shape block showing different surface finishes.

FIG. 14 is a perspective view of a game board in accordance with the present invention, with a die.

### DETAILED DESCRIPTION

Various apparatuses or methods will be described below to provide an example of an embodiment of each claimed invention. No embodiment described below limits any claimed invention and any claimed invention may cover apparatuses or methods that differ from those described below. The claimed inventions are not limited to apparatuses or methods having all of the features of any one apparatus or method described below or to features common to multiple or all of the apparatuses described below. It is possible that an apparatus or method described below is not an embodiment of any claimed invention. The applicants, inventors and owners reserve all rights in any invention disclosed in an apparatus or method described below that is not claimed in this document and do not abandon, disclaim or dedicate to the public any such invention by its disclosure in this document.

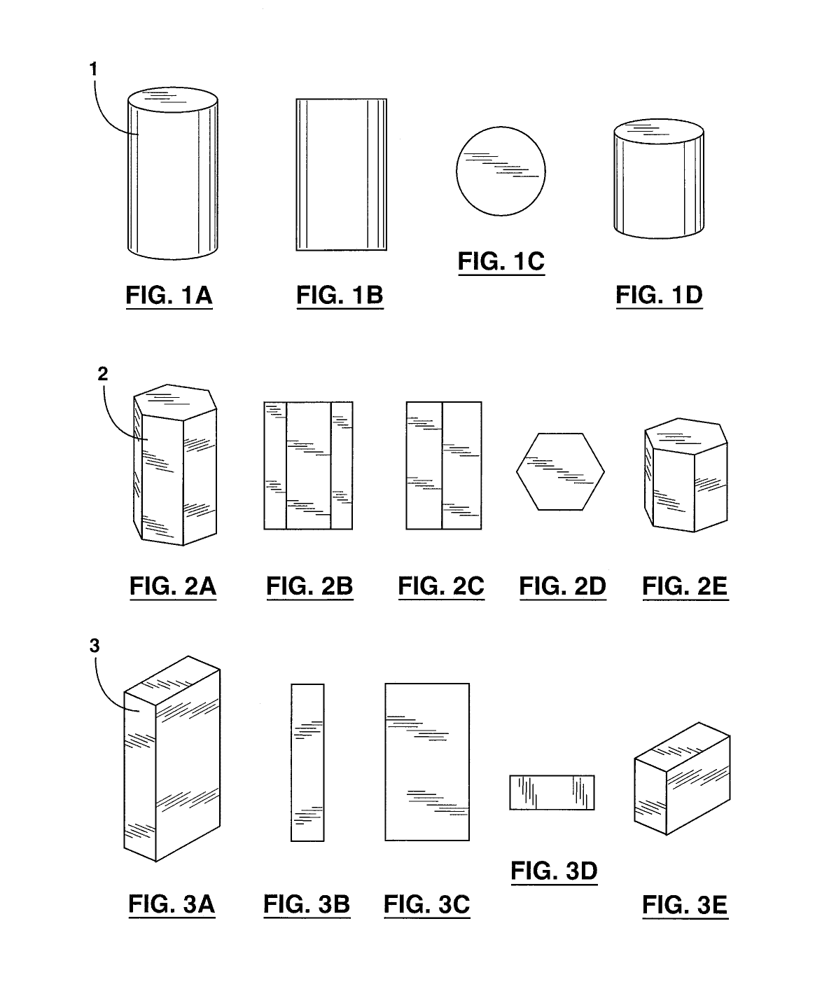

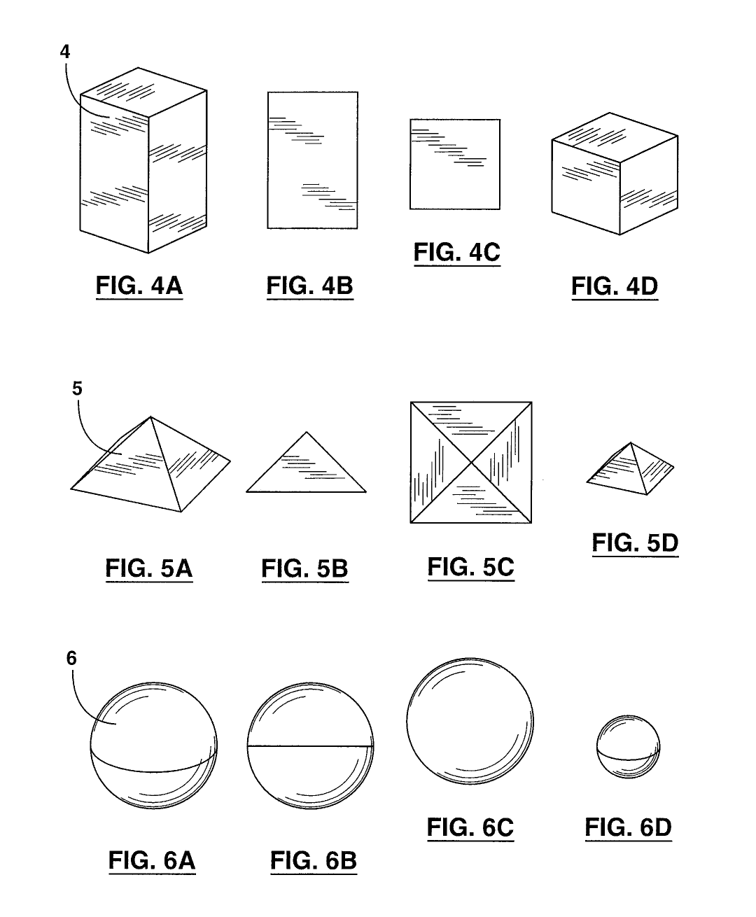

FIGS. 1-7 provides a representation of seven blocks in accordance with the present invention which have different shapes, but it will be understood that the invention is not limited to the specific shapes shown. The blocks shown comprise: a cylindrical column 1; an hexagonal column 2; a rectangular prism 3; a square column 4; a pyramid 5; a sphere 6; and a triangular prism 7. As shown, each of these blocks 1-7 has both large and small configurations. For the blocks 1, 2, 3 and 4, this is achieved by changing one dimension of the shape, which can be identified as a height of the block, but keeping the cross-section of each of these blocks unchanged. For the blocks 5 and 6, the change in shape is achieved by keeping the overall shape the same, but changing all the dimensions by the same amount. For the block 7, two dimensions are changed and one kept constant, to effect the change in shape. Exemplary dimensions are shown in FIGS. 1-7.

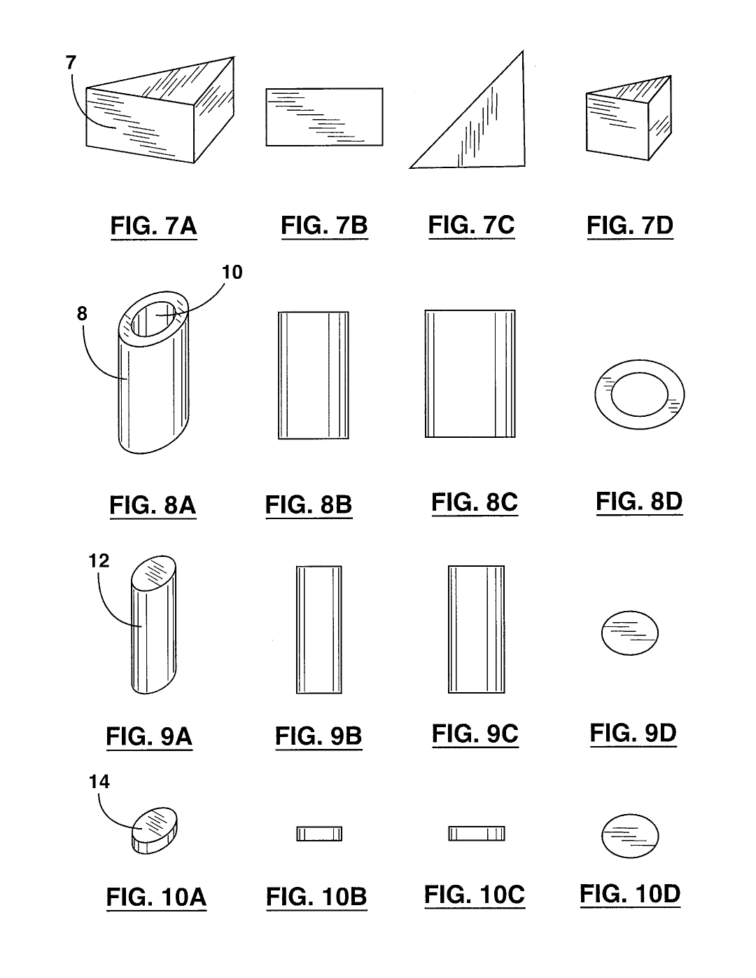

It is also possible for each block to be made in three or more different sizes. Additionally, the different sizes, generally, can be achieved by changing one, two or three different dimensions. It will be understood that the shape of the block may constrain the number of dimensions that can be changed; e.g. for a spherical or cubic block, all three dimensions have to be changed by the same amount; for a triangular prism one can change just the depth of the block, e.g as in FIG. 7, both dimensions giving the triangular shape by the same amount, or all three dimensions together.

Depending on the intended use of the blocks, different sizes can be provided in different ways. If the blocks are intended mainly for sorting, either as a game or as a diagnostic technique, it may be preferable for all the blocks to vary in size by changing all three dimensions, so that, for each shape, the different sizes are identical shapes. If the blocks may be used for building and the like, at least for blocks with parallel end faces, it may be preferable to keep the cross section of the block constant and to vary the length or third dimension, e.g. as shown in FIGS. 1A-C and FIG. 1D, and in FIGS. 2A-D and FIG. 2E, with the different lengths being an integral multiple of one another.

Consequently, the blocks in FIGS. 1-7 display two of six sorting categories: shape and size. Other sorting categories are colour, texture, and finish will be applied onto the surface of the blocks detailed in FIGS. 1-7. A further category for the blocks is weight, i.e. the blocks may either be categorized as heavy or light. This listing of categories may not be exhaustive, and other categories may be possible.

Texture can encompass smooth, rough, soft, fibrous and other characteristics. The surface of the blocks can be covered with woven or knitted fabric to give different textures, with, for example, fabric formed from threads of different thicknesses to affect the texture. The fabric can simulate a feathered or fur covered surface. For the finish category, this can include glossy, flat or matte, or a variety of intermediate finishes.

Colour can include the primary colours red yellow and blue, and optionally black and white. The colours may also include, for example, orange, green and violet.

Different weighting of the blocks can be achieved in a variety of different ways. The blocks may be formed from wood. Then, light blocks may require no modification from the basic wooden block. Heavy blocks can then be provided by modifying each light, wooden block, by the addition of a denser weight element, that does not change the shape of the block. FIGS. 8-11 shows one way of adding weight to a block. FIGS. 8-11 shows the addition of weight to a large cylindrical or elliptical Column 8, but the method is applicable for all shapes of blocks 1-7. In particular, while FIGS. 8-11 show a Column that is an elliptical, the technique shown is readily applicable to a cylindrical Column. For cylindrical Column, the board formed in the block would correspondingly be cylindrical, and thus can be readily formed by a variety of different methods, including drilling.

The method comprises forming a hole by boring into a block 8. This leaves an elliptical or cylindrical bore hole 10. A rod 12 made of a material denser than the original material of the block 8 which may be wood, and the same profile as the bore hole 10 (to permit insertion into the hole 10) is cut such that the remaining rod's length is approximately 90% of the depth of the bore hole. The rod 12 may be formed of steel. A wooden dowel or plug 14 with the same diameter or shape as the bore hole 10 is cut such that the remaining dowel's length is approximately 10% of the depth of the bore hole 10, or slightly greater. The cut rod 12 is inserted into the bore hole 10. The cut wooden dowel 14 is then inserted into the bore hole 10, with a bonding medium such as wood glue added to insure the dowel holds fast. The remaining surface is sanded down so that the wooden dowel is flush with the original block face. The block is then painted and finished, leaving no trace of the modifications.

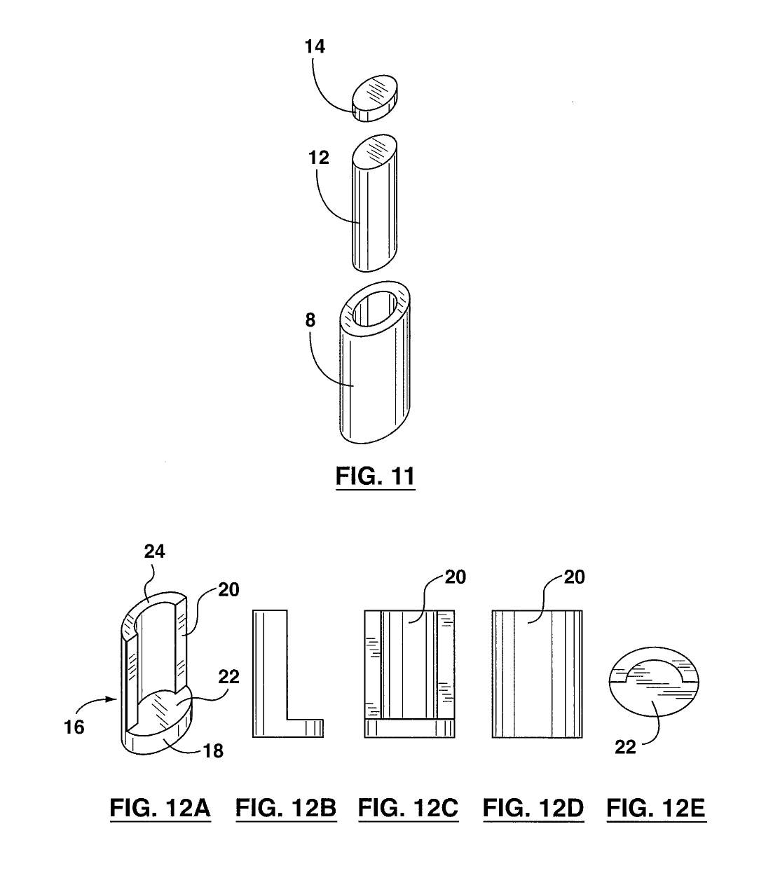

FIG. 12 shows an alternative way of assembling a block. An element 16 is provided that forms half of a block. The element 16 has a base 18 and a side wall 20 with an end surface 24. The base 18 has an inner surface 22. Two elements 16 can be assembled together with their inner surfaces and end faces abutting one another. This will then define a cavity as for the block of FIG. 11, which can be filled with a weight in the same manner as FIG. 11 or otherwise.

It will be understood that the blocks 1-7 can be made by a variety of techniques and from a variety of materials. For example the blocks could be molded as hollow shapes from a plastic material. Hollow, plastic blocks would then be light blocks, and to form heavy blocks, each block can be filled, or partially filled, with a material of a different density, to give a desired weight. Possibly two different fill materials can be used. As noted texture and surface finish can be changed by adding surface layers to the blocks, e.g. of fabric.

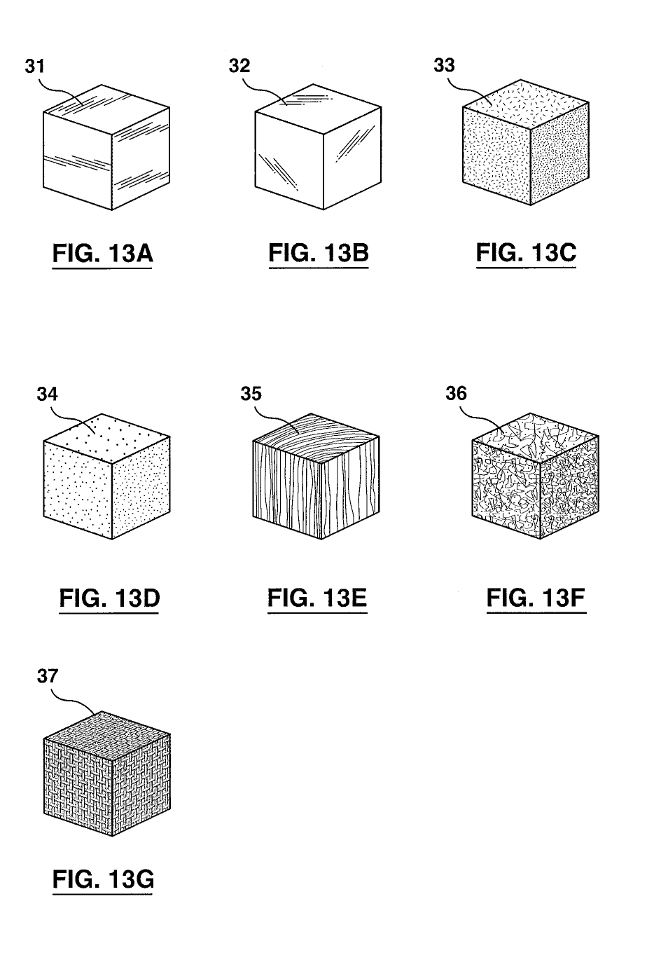

FIGS. 13A-G show, by way of example, different surface treatments for a cubic block, but applicable to any shape or size of block. Block 31 has a smooth/plastic/metal finish, block 32 has a gloss/plastic/metal finish, and block 33 has a matte/rough finish. Blocks 34, 36 and 37 show respectively soft, fibrous and knitted or woven textures. Block 35 shows a wood finish.

The set of blocks may be designed for use by children with neurological disorders, such as autism their parents, and their caregivers. The blocks are also intended for use with the children or other individuals with cognitive or neurological disorders that may have difficulty with categorization, e.g. dyslexia, visual memory. The blocks may be used both to diagnose neurological or other disorders, and as a rehabilitation tool. Additionally, the blocks may find use as a learning aid or simply a play item for children or individuals with no neurological or other deficits. The set of blocks may also be designed to be used in a single or multi-player game. The set of blocks can be categorized by a variety of characteristics. These characteristics or features may be ordered from least abstract to most abstract, as follows:

1. Shape: This is the most basic characteristic. The wooden blocks can be made in a variety of shapes. Specifically, the game envisions the use of seven shapes:
a. Triangular Prisms
b. Square Columns
c. Circular Columns
d. Rectangular Columns
e. Spheres
f. Pyramids
g. Hexagonal Columns

2. Size: Each shape of block can be made in one of two sizes: (a) large, (b) small.

3. Colour: Each block can be painted in one of a selected number of colours, e.g. one of three colours. These colours may be chosen to enhance enjoyment while minimizing unwanted reactions due to negative visual stimuli associated with certain colours. The colours chosen may be blue, green, and orange. Possibly also different patterns, such as striped, checked, etc. can be used

4. Finish: Each block may appear in one of two or more selected finishes, e.g.: (a) matte or (b) reflective or gloss.

5. Texture: Each block maybe one of a selected number of textures, e.g. one of three textures: (a) smooth, (b) rough, and (c) fibrous.

6. Weight: Each block may appear in one of two weights: (a) heavy or (b) light. It may also be possible to provide blocks with three or more different weights where it is possible to make these weights sufficiently different that they can be sensed.

It will be understood that, covering all these possible combinations of characteristics may give too many different blocks. From the example above the total number of different combinations is 7×2×3×2×3×2=504. This can be reduced by arbitrarily not including all possible combinations, since for either a game or for diagnosis it is not necessary to include all possible combinations. Alternatively, one can reduce the number of characteristics, i.e. instances in each category, and then include all possible combinations, e.g. with: 4 different shapes, or instances in the shape category; 2 sizes; 2 colours; 2 finishes; 2 textures; and 2 weights, for a total number of different combinations of 128. Another possibility is to omit one characteristic type entirely, e.g. omit any variation in texture and/or finish, to keep the total number of possible combinations reasonable.

The present invention also provides games and sets of rules for the games to be played with the blocks. To play these games, in addition to the blocks 1-7, there is also provided one or two dice. Each die has a number of faces that correspond to the number of different categories detailed above. If the number of categories is less than 6, then one or more categories can be repeated on the die faces. If the number of categories is greater than six, then a die with more than six faces can be provided and/or some categories can be repeated on the die faces.

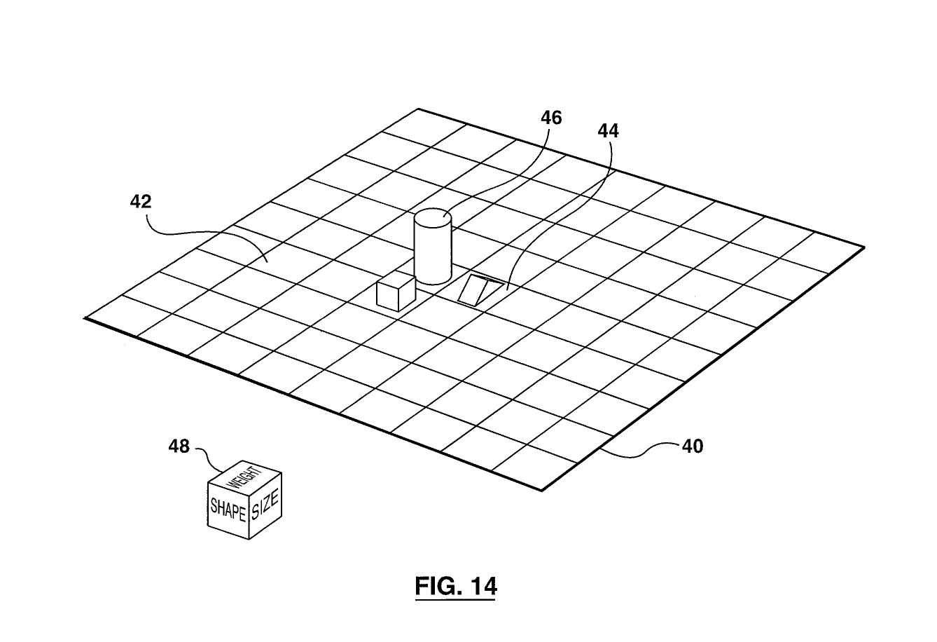

To play a game, a game board can be used, and an exemplary game board is shown in FIG. 14 with a playing surface 40. The playing surface 40 defines individual playing squares 42 or areas. A grid pattern is provided with an odd 9×9 matrix, so that there is a central square or playing space 44, which can be specially marked and the rules can require that the first block played be placed into the square. FIG. 14 also shows blocks 46 placed on the playing surface 40. A six sided died, as described above, is shown at 48. As indicated, the six faces of the die can each indicate different categories for the blocks, e.g. shapes, size, weight, etc.

Exemplary games are described below. It will be understood that these games could be played without a game board, somewhat in the matter of the game of dominos. However, as the blocks of the invention may have a variety of different shapes and sizes, it may be preferable to use a game board having defined squares 42, for simplicity and clarity.

The rules for the single player game are as follows. An activity leader rolls one of two provided dice. As above, the six sided die may have one of the six categories on each face. The player is asked to sort the blocks by the category rolled.

### Single Player (Autism Diagnosis)

To use the blocks as a diagnostic tool, one applies the same methods as described by the Wisconsin card sorting test, and no die is needed. Thus the child is asked to sort the blocks, without telling him or her what is the sort criterion. The child is given feedback as to whether they are sorting correctly or not. At various times the sort criterion is changed, without telling the child, and the mistakes made in learning the new sort criterion and how long it takes to learn it are noted.

### Two Player Game

The rules for the two player game are as follows:

1. Each player takes half the blocks.

2. One player, who may be the youngest, places a block of their choosing on one of the squares 42 on the playing surface on board 40. Rules may require that the first block be placed in a central square 44. Blocks placed on the playing surface are indicated at 46. Each square 42 of the matrix is a valid playing location, for a total of eighty one playable spaces.

3. Each block 46 on the playing surface 40, or the square 42 in which it is placed, is considered to have four playable faces, which can be identified as North, East, South, and West respectively, irrespective of the exact shape of the block.

4. The second player rolls the die 48. The second player must match one of his or her blocks to any of the blocks on the playing surface by the category rolled. Once matched, the two playable faces of the matched blocks are “occupied” and cannot be used to form another match. The remaining playable faces may still be matched to a third block. Thus if the rolled die shows “colour” on its top surface, the second player has to play a block of the same colour as one already played.

5. Each player takes a turn rolling the die and attempting to match blocks as dictated by the die. Each block played must match a previously played block according to the category indicated by the die roll

6. If a player a places block such that multiple blocks can be involved in a match, all of the blocks must match according to the category stipulated by the dice. Continuing with the example of colour, if a player places a block on the playing surface which comes in contact with two different blocks, all three blocks must be the same colour. This is called a multi-match. Achieving a multi-match earns the player a free roll of the dice. There may then be some strategy in playing blocks not in a straight-line, but adjacent so that the playable faces, or edges of squares 42, border a common and unoccupied playing square 42. This then requires that block played on that unoccupied square match all the two or more adjacent blocks, as described.

7. The first player to use all of their blocks wins.

### Variation: Two Player Game (Challenging)

For a more challenging version of the two player game, the players may opt to play with both of the dice provided. The rules are identical to the above, except when placing a block next to one already on the playing surface, the block must match according to both categories shown by the dice. A double matching block must also match the two die features to the two different adjacent blocks, and in some circumstances this may be impossible.

### Variation: More Than Two Players

The game can also be played by more than two players, if there are a sufficient number of blocks. For larger number of players, it may be desirable to provide more than one of each type of block.

While the games above have been described as being suitable for children with neurological disorders, it will be understood that they can be played by anyone. In particular, the more challenging two player version may be enjoyable for play by children or individuals with no neurological challenges or deficits.

Other refinements are possible. Many of the rules of dominoes can be incorporated into the game. For example, a rule could limit which faces a block can be used to form a match, so that the blocks can only be played extending in line. At the start of play not all the blocks could be distributed to the players. Each player could start with the same number of blocks, with some held in a common stockpile. Further, in the event that a player cannot play, they can be required to take a block from the stockpile.

The present invention also may have application to domino-type games. In this game mode, game pieces are provided with indicia, commonly spots, that are only present on one face and can be hid, by turning the game pieces upside down. The game pieces, according to the present invention can have two or more different characteristics or features selected from different categories, to enable more complex games to be played. Some or all of these features can be on just one face so they can be hidden. For example the face with the spots can be provided with different colours, or different textures.

### REFERENCES

The following documents are referenced, and content of each of these is hereby incorporated by reference.
[1] National Institute of Neurological Disorders and Stroke (NINDS), “Autism Fact Sheet,” tech.rep., National Institute of Neurological Disorders and Stroke(NINDS), 2009.
[2] L. Klinger and G. Dawson, “Prototype formation in autism,” Development and Psychopathology, vol.13, no.01, pp.111-124, 2001.
[3] D. Bowler, N. Matthews, and J. Gardiner, “Asperger's syndrome and memory: Similarity to autism but not amnesia,” Neuropsychologia, vol.35, no.1, pp.65-70, 1997.
[4] N. Minshew, G. Goldstein, and D. Siegel, “Neuropsychologic functioning in autism: Profile of a complex information processing disorder,” Journal of the International Neuropsychological Society, vol.3, no.04, pp.303-316, 1997.
[5] T. Grandin, Thinking in pictures: And other reports from my life with autism. Vintage, 2006.
[6] R. Heaton, A manual for the Wisconsin card sorting test. Western Psychological Services, 1981.
[7] C. Johnson, S. Myers, et al., “Identification and evaluation of children with autism spectrum disorders,” AAP Policy, vol.120, no.5, p.1183,2007.
[8] D. Napolitano, T. Smith, J. Zarcone, K. Goodkin, and D. McAdam, “Increasing Response Diversity in Children with Autism.,” Journal of Applied Behavior Analysis, vol.43, no.2, p.7, 2010.
[9] G. Merrett and R. Stewart, “The Design of Toys for Autistic Children,” Studies in Design Education Craft & Technology, vol.9, no.2, 2009.
[10] Fine Wooden Toys, “Classroom Unit Blocks.” Online: http://www.finewoodentoys.com/pro1059030.html
[11] A. Ludlow, A. Wilkins, and P. Heaton, “The effect of coloured overlays on reading ability in children with autism,” Journal of autism and developmental disorders, vol. 36, noA, pp.507-516, 2006.

1. A set of blocks, for playing a game or for diagnosis, the set of blocks comprising a plurality of blocks, each block having one characteristic selected from a first category and one characteristic selected from a second category, wherein at least some of the characteristics in the first category are different from one another, and at least some of the characteristics in the second category are different from one another.

2. A set of blocks as claimed in claim 1, wherein each block includes at least one characteristic from at least three different categories.

3. A set of blocks as claimed in claim 2, wherein within the set of blocks for each of the three categories, there are at least two different instances of that category.

4. A set of blocks as claimed in claim 2, wherein each block includes one characteristic selected from each of six categories.

5. A set of blocks as claimed in claim 4, wherein within the set of blocks for each of the six categories, there are at least two different instances of that category.

6. A set of blocks as claimed in any one of claims 1 to 5, wherein the categories include varying degrees of abstraction.

7. A set of blocks as claimed in claim 6, wherein differences in at least one category that cannot be determined by sight alone.

8. A set of blocks as claimed in claim 4 or 5, wherein the categories comprise shape, size, colour, texture, with weight and surface finish.

9. A set of blocks as claimed in claim 8, wherein the category of texture includes at least one of smooth, rough, soft, fibrous, woven, knitted characteristics.

10. A set of blocks as claimed in claim 9, wherein the category of surface finish includes at least a matte finish and a gloss finish.

11. A set of blocks as claimed in any one of claims 1 to 10, wherein the blocks are formed from at least two different materials having different densities, to enable the blocks to be provided with different weights.

12. A set of blocks as claimed in claim 11, wherein the blocks are formed from wood and a metal, preferably steel.

13. A set of blocks as claimed in any one of claims 1 to 11, wherein the blocks are hollow and formed from plastic, and to provide different weights for the blocks, with at least some hollow, plastic blocks filled with another material to give a different weight.

14. A method of playing a game, preferably for a child with a neurological disorder, the method comprising:
providing a plurality of blocks as claimed in any one of claims 1 to 13, and a die bearing on its faces an indication of each of the different categories of features of the blocks;
rolling the die, to select a category, or otherwise randomly selecting one of the categories; and
asking the player of the game to sort the blocks according to the selected category.

15. A method of diagnosing autism in a child, the method comprising:
providing a plurality of blocks as claimed in any one of claims 1 to 11;
asking the child to sort the blocks according to an unspecified category, comprising a sort criterion;
providing feedback to the child as to whether blocks are sorted correctly or not; and periodically changing the category by which blocks are to be sorted, while continuing to provide feedback as to whether the blocks are sorted correctly or not; and noting mistakes made in sorting and how long it takes the child to learn a new sort criterion.

16. A method of playing a game with two people, preferably two children with neurological disorders, the method comprising:
providing a plurality of blocks as claimed in any one of claims 1 to 13, dividing the blocks between the two players, and providing a die bearing on its faces an indication of each of the different categories of features of the blocks;
having a first player select and place a block on a playing surface; rolling the die, to select a category, or otherwise randomly selecting one of the categories, and the second player then playing a block by placing it on the playing surface adjacent the first block, with the second block having the same feature from the selected category as the first block;
repeating the last step for each player in turn.

17. A method of playing a game as claimed in claim 14, including providing a game board, a playing surface, and a grid pattern provided on the playing surface dividing the playing surface into individual playing areas, wherein the method includes each player placing a block in a selected playing area.

18. A method as claimed in claim 17, wherein the game board is provided with a grid pattern comprising an odd number of rows and columns, to define a central playing area, which is designated as a start area, which the first block must be placed.

19. A method as claimed in claim 16, 17, or 18, including providing for blocks to be played so that two or more blocks border on a common and an occupied playing area, and requiring that a block placed on the common and an occupied playing area must have the same feature from the selected category as all the blocks bordering the common and unoccupied playing area

20. In combination, a set of blocks as claimed in any one of claims 1-11 and a game board and at least one die, for playing a game.
